(19) United States
(12) Patent Application Publication
(10) Pub. No.: US 2025/0322902 A1
(43) Pub. Date: Oct. 16, 2025

(54) MULTI-OBJECTIVE REINFORCEMENT LEARNING WITH EXPERIMENTAL FEEDBACK FOR PROTEIN DESIGN

(71) Applicant: UCHICAGO ARGONNE, LLC, Chicago, IL (US)

(72) Inventors: Arvind RAMANATHAN, Naperville, IL (US); Gautham DHARUMAN, Westmont, IL (US); Heng MA, Downers Grove, IL (US); Priyanka Varadaraja SETTY, Aurora, IL (US); Logan Timothy WARD, Brookhaven, GA (US); Ozan GOKDEMIR, Chicago, IL (US); Alexander BRACE, Willowbrook, IL (US); Kyle HIPPE, Chicago, IL (US); Anima ANANDKUMAR, Pasadena, CA (US)

(73) Assignee: UCHICAGO ARGONNE, LLC, Chicago, IL (US)

(21) Appl. No.: 19/174,661

(22) Filed: Apr. 9, 2025

Related U.S. Application Data
(60) Provisional application No. 63/632,977, filed on Apr. 11, 2024.

(51) Int. Cl. G16B 15/20; G06N 20/00; G16B 40/20

(52) U.S. Cl. CPC G16B 15/20; G06N 20/00; G16B 40/20

(57) ABSTRACT

A method for designing proteins using multi-objective reinforcement learning can include generating, by one or more processors using a machine model, based on an initial protein sequence data structure, a plurality of protein sequences, the machine learning model configured based on reinforcement learning from a plurality of reward metrics including at least one reward metric associated with experimental data regarding example sequence data, scoring, by the one or more processors, using a plurality of scoring functions, the plurality of protein sequences, to select a subset of protein sequences of the plurality of protein sequences, and outputting one or more selected protein sequences of the subset of selected protein sequences.

### CROSS-REFERENCE TO RELATED APPLICATIONS

The present application claims priority to U.S. Provisional Patent Application No. 63/632,977, filed on Apr. 11, 2024, the disclosure of which is incorporated herein by reference in its entirety and for all purposes.

### FIELD OF DISCLOSURE

The present disclosure relates generally to a multi-objective reinforcement learning model. Specifically, the current disclosure relates to systems and methods for designing and generating protein or genome sequences.

### SUMMARY

At least one aspect of the present disclosure relates to a method. The method can include generating, by one or more processors using a machine model, based on an initial protein sequence data structure, a plurality of protein sequences, the machine learning model configured based on reinforcement learning from a plurality of reward metrics including at least one reward metric associated with experimental data regarding example sequence data. The method can include scoring, by the one or more processors, using a plurality of scoring functions, the plurality of protein sequences, to select a subset of protein sequences of the plurality of protein sequences and outputting one or more selected protein sequences of the subset of selected protein sequences.

In some implementations, the machine learning model can include a language model. The method can determine, by the machine learning model, each protein sequence of the plurality of protein sequences. The machine learning model can include generating one or more protein sequence elements based on the initial protein sequence data structure. The machine learning model can be configured based on reinforcement learning by a plurality of agents, each agent of the plurality of agents associated with a different reward metric of the plurality of reward metrics than each other agent of the plurality of agents. The machine learning model can include a pre-trained language model fine-tuned based on the plurality of reward metrics.

In some implementations, the plurality of scoring functions can include at least a similarity function based on a database of example sequence data, a folding function, and a stability function. The method can include scoring using the stability function responsive to an output of at least one of the similarity function or the folding function satisfying a corresponding threshold. Generating the plurality of protein data sequences can include generating a plurality of protein sequence data elements for each protein data sequence of the plurality of protein data sequences. Each protein sequence data element of the plurality of protein sequences data elements can represent at least one of a codon or a protein residue. The plurality of scoring functions can include at least one function based on at least one of a guanine-cytosine (GC) content or a molecular weight of each protein sequence of the plurality of protein sequences.

In some implementations, the plurality of reward metrics can include at least one evolutionary conservation metric. The plurality of reward metrics can include at least one molecular simulation metric. The method can include asynchronously performing, by the one or more processors in parallel using a plurality of parallel computing resources, at least one of the generating of the plurality of protein sequences or the scoring of the plurality of protein sequences. The plurality of scoring functions can include an activity function to determine an activity of at least one protein sequence of the plurality of protein sequences.

At least one aspect of the present disclosure relates to a system. The system can include one or more processors. The one or more processors can generate, using a machine model, based on an initial protein sequence data structure, a plurality of protein sequences. The machine learning model can be configured based on reinforcement learning from a plurality of reward metrics including at least one reward metric associated with experimental data regarding example sequence data. The one or more processors can score, using a plurality of scoring functions, the plurality of protein sequences, to select a subset of protein sequences of the plurality of protein sequences. The one or more processors can output one or more selected protein sequences of the subset of selected protein sequences.

In some implementations, the machine learning model of the system can include a language model. The one or more processors can determine each protein sequence of the plurality of protein sequences by generating, using the language model, one or more protein sequence elements based on the initial protein sequence data structure. The machine learning model can include a pre-trained language model fine-tuned based on the plurality of reward metrics. The plurality of reward metrics can include at least one evolutionary conservation metric and at least one molecular simulation metric.

In some implementations, the plurality of scoring functions can include at least a similarity function based on a database of example sequence data, a folding function, and a stability function. The plurality of scoring functions can include scoring using the stability function responsive to an output of at least one of the similarity function or the folding function satisfying a corresponding threshold. The one or more processors can include a plurality of parallel processing units to asynchronously perform at least one of the generation of the plurality of protein sequences or the scoring of the plurality of protein sequences.

At least one aspect of the present disclosure is directed towards a method. The method can include generating, by each of a plurality of reinforcement learning agents, for each protein sequence of a plurality of examples of protein sequences, a reward score for an objective function. The reward score can be generated based on a different metric for each agent of the plurality of reinforcement learning agents. The method can include evaluating the objective function using each reward score to generate an output of the objective function. The method can include updating a language model based on the output,

In some implementations, the metric of a first agent of the plurality of reinforcement learning agents can correspond to a structure of the protein sequence, and the metric of a second agent of the plurality of reinforcement learning agents can correspond to kinetics of the protein sequence. Updating the language model can include evaluating a Kullback-Leibler divergence with respect to a previous state of the language model.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

### BRIEF DESCRIPTION OF DRAWINGS

FIG. 1 depicts a schematic diagram of an example of a system for designing and generating protein sequences.

FIG. 2 depicts a schematic diagram of an example of a multi-objective reinforcement learning (MORL) model.

FIG. 3 depicts a schematic diagram of an example of a reinforcement learning (RL) loop for a single iteration of finetuning a large language model (LLM).

FIG. 4 depicts a schematic diagram of an example of a method for designing and generating protein sequences.

FIG. 5 is a chart of an example of task scheduling and resource allocation for execution of one or more processes for protein sequence generation on a graphics processing unit (GPU) of a software environment.

FIG. 6 is a table of an example of a computational performance of a model in mixed precision (MP) with different model sizes and a sequence length of 2,048.

FIG. 7 is a table of an example of a final loss and perplexity values achieved by the (MORL) model for a sequence length of 2,028 and 10,240.

FIG. 8 is a table of an example of a throughput and training time of a Cerebras Wafer-Scale Cluster for the (MORL) model for a sequence length of 10,240.

FIG. 9 is a table of an example of metrics of the (MORL) model using the Cerebras Wafer-Scale Cluster on a sequence length of 10,240.

### DETAILED DESCRIPTION

Following below are more detailed descriptions of various concepts related to, and implementations of methods and systems for designing protein sequences. The various concepts introduced above and discussed in greater detail below may be implemented in any of numerous ways as the described concepts are not limited to any particular manner of implementations. Examples of specific implementations and applications are provided primarily for illustrative purposes.

While the current disclosure describes the design of protein sequences as being performed by a multi-objective reinforcement learning model, it is to be understood that the methods described herein can be implemented or integrated within a system that may perform a combination of training, generating, scoring, and evaluating of protein sequences.

Designing complex biological systems (e.g. proteins, peptides, biological pathways, genomes) holds promise in transforming and advancing a number of applications (e.g., biomedicine, biomanufacturing, biomaterials, synthetic biology). While computational processes can be used for designing such systems (e.g., generating protein sequences), the search space for the design process can be vast (e.g., n²⁰, where n represents the number of amino acids of the sequence), even accounting for the number of possible protein sequences in the search space that would not be expected to have any function. Moreover, designing proteins can present specific challenges. For example, within a biological pathway, an enzyme may have a desired catalytic activity, which can be measured by its k_{cat} which affects downstream interactions, and eventually production of the desired product. While various rules-based and/or machine learning-based technologies can be used to streamline the protein design process, challenges may arise for such technologies to operate effectively under constraints of computational resources as well as meeting criteria for the characteristics of the proteins. For example, some systems of designing proteins rely on extensive mutational experiments by either random modifications to a gene of interest, rule-based computational design, or emerging AI-enabled techniques (e.g. LLMs) or diffusion approaches. Random mutations may explore a potentially complex and large design space. Thus, there may be little opportunity for feedback from simulations or experiments as the generative modeling. For examples, modifying 50 amino acid positions within an enzyme with a sequence length of 300 amino acids implies, for example, 50e300 approaches which can use extensive time and computational power and lacks the ability to incorporate iterative feedback as the model modifies the amino acid positions resulting in less than desirable results.

Some other systems use directed evolution and/or extensive integration of computational approaches (e.g., Monte Carlo methods) with experimental techniques to design protein sequences, but such techniques can be too computationally intensive to be deployed effectively. While some systems use natural language processing or generative adversarial networks, such systems can lack insight or feedback from experiments or generative sequences and may have hallucinatory effects (e.g., generating sequences that resemble functional protein sequences that are invalid). Other systems may use 3-letter codon-based representation for protein sequences in models that learn evolutionary trajectories of viral genomes. However, these systems lack a comprehensive iterative feedback system in generating protein sequences.

Systems and methods as described herein can enable the design of proteins with specific properties and bioprocesses (e.g. pathways) using an iterative workflow that can leverage large language models (LLMs) at the gene and protein level while incorporating multiple levels of feedback. Systems and methods performed in accordance with the present solution can enable multi-objective reinforcement learning to generate, design protein and genome sequences by implementing multiple objectives through iterative reinforcement learning loops. As described herein, systems and methods in accordance with the present solution can be implemented to solve a variety of computational problems, including but not limited to, designing proteins, designing genomes, expanding a design space beyond modifying existing proteins, developing vaccines, novel gene therapies, and target protein-protein interactions. For example, a plurality of criteria for evaluating (e.g., scoring) candidate protein sequences can be executed in a prioritized manner tied to both biological factors and computational resource management to more efficiently generate high quality protein sequences. Parallel processing and/or task optimization, including parallel execution of different tasks in the protein design processes, can be implemented to facilitate more efficient computational resource usage. Systems and methods as described herein can be scaled and generalized to include other proteins (e.g., new), biomolecules, and bioprocesses (e.g., pathways). Various such functionalities of systems and methods described herein can allow for computational protein design to be tractable, e.g., to achieve generation of protein designs that are valid and satisfy target metrics, given available computational resources.

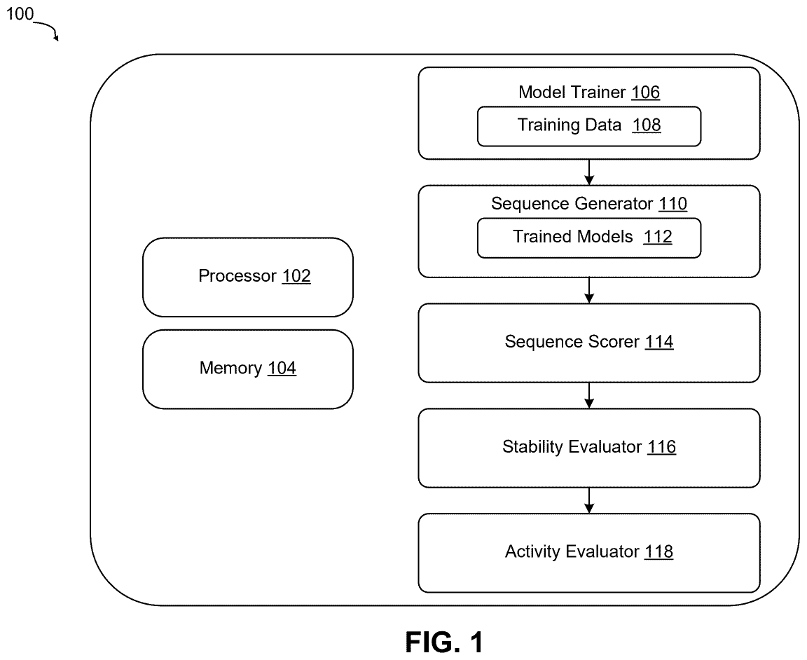

FIG. 1 depicts a system 100 for a multi-objective reinforcement learning (MORL) approach for protein design incorporating experimental data. The system 100 includes one or more processors 102 and memory 104, which can be implemented as one or more processing circuits. The processor 102 may be a general purpose or specific purpose processor, an application specific integrated circuit (ASIC), one or more field programmable gate arrays (FPGAs), a group of processing components, or other suitable processing components. The processor 102 may be configured to execute computer code or instructions stored in memory 104 (e.g., fuzzy logic, etc.) or received from other computer readable media (e.g., CDROM, network storage, a remote server, etc.) to perform one or more of the processes described herein. The memory 104 may include one or more data storage devices (e.g., memory units, memory devices, computer-readable storage media, etc.) configured to store data, computer code, executable instructions, or other forms of computer-readable information. The memory 104 may include random access memory (RAM), read-only memory (ROM), hard drive storage, temporary storage, non-volatile memory, flash memory, optical memory, or any other suitable memory for storing software objects and/or computer instructions. The memory 104 may include database components, object code components, script components, or any other type of information structure for supporting the various activities and information structures described in the present disclosure. The memory 104 may be communicably connected to the processor 102 and may include computer code for executing (e.g., by processor 102) one or more of the processes described herein. The memory 104 can include various modules (e.g., circuits, engines) for completing processes described herein. The one or more processors 102 and memory 104 may include various distributed components that may be communicatively coupled by wired or wireless connections; for example, various portions of system 100 may be implemented using one or more client devices remote from one or more server devices. The system 100 can include any one or more rules, heuristics, logic, code, functions, machine learning models, neural networks, algorithms, or various combinations thereof to implement one or more components of the system 100, such as any one or more of model trainer 106, training data 108, sequence generator 110, models 112, sequence scorer 114, stability evaluator 116, and activity evaluator 118. The system 100 and/or various components thereof can execute various operations described herein and/or combinations thereof as one or more tasks. For example, the model trainer 106 can cause the processor 102 to execute a training task; the sequence generator 110 can cause the processor 102 to execute a sequence generation task.

### Reinforcement Learning for Protein Sequences Generator Models

The system 100 can include a model trainer 106. The model trainer 106 can be used to train various machine learning models described herein (e.g., models 112), such as to provide training data as input to a machine learning model, cause the machine learning model to generate estimated outputs responsive to the inputs, and update the machine learning model (e.g., one or more parameters of the machine learning model) according to an evaluation of the estimated outputs. For example, the model trainer 106 can perform unsupervised and/or supervised learning processes to update the machine learning model; this can include, for example, using any of various objective functions and/or cost functions to evaluate the estimated outputs of the machine learning model, and techniques including but not limited to gradient descent to perform the updating of the machine learning model.

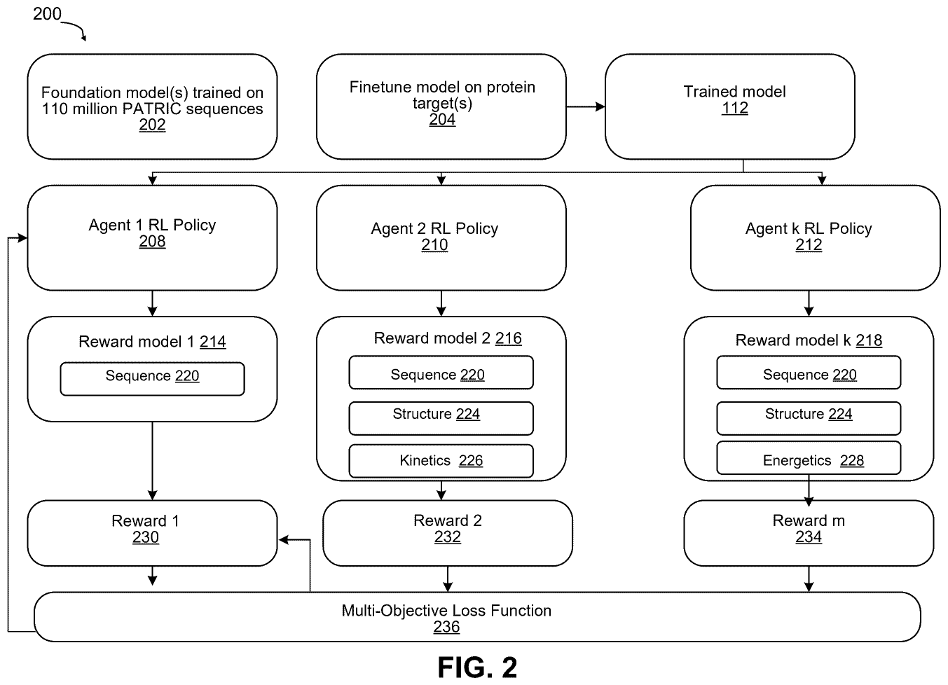

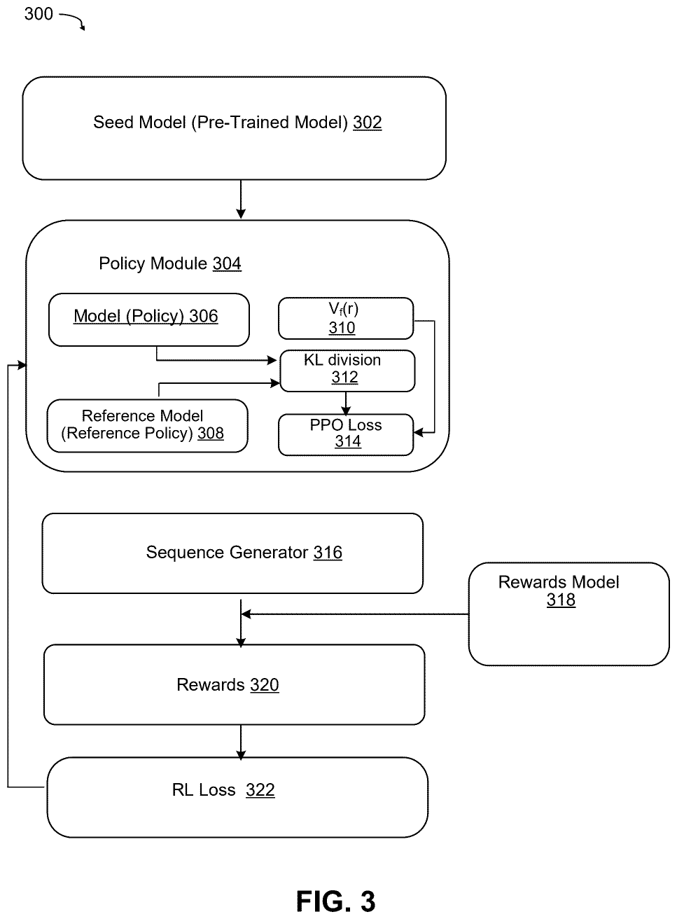

The model trainer 106 can perform reinforcement learning to train a reinforcement learning (RL) model, such as a MORL model. For example, the model trainer 106 can train the RL model to cause the RL model to learn a policy for performing actions with respect to rewards, such as actions for generating or modifying protein sequences according to the rewards. The model trainer 106 can incorporate features of system 200 described with reference to FIG. 2 and/or system 300 described with reference to FIG. 3.

The model trainer 106 can update the RL model based on feedback regarding outputs of the model. The feedback can include rewards or penalties, and can be representative of performance of the RL model with respect to factors such as the protein sequences outputted by the RL model and experimental data. The feedback can be numerical signals based on a performance of the RL model. The feedback can be training feedback. The model trainer 106 can use the feedback to update parameters of the RL model to minimize a difference between (predicted) outputs of the RL model and true targets, e.g., experimental data. The feedback can be a loss or error metric. The model trainer 106 can use iterative feedback which can be provided simultaneously, concurrently, or sequentially.

In some implementations, the model trainer 106 updates the RL model based on evaluation of an objective for the RL model to achieve during a training process. For example, the model trainer 106 can evaluate, based on the output of the RL model and/or feedback regarding the output, an objective that includes a stability criterion with respect to a genome sequence represented by the output. The stability criterion can be satisfied by the sequence being resistant to changes or disruptions to its structural integrity and biological function. The stability criterion can be based on a Gibbs free energy difference between a folded and unfolded protein sequence.

The system 100 can include one or more models 112, such as machine learning models and/or RL models. The models 112 can include language models. The models 112 can include LLMs. The models 112 can include genome-scale language models (GenSLMs). A GenSLM can be a genome scale foundation model which can be generalized to other prediction tasks, e.g., not genomes or proteins. The GenSLM can overcome limitations of a pre-trained language model (PLM). Limitations can include lacking iterative feedback. GenSLMs can use 3-letter codon-based representation for protein sequences. GenSLMs can better capture and enable much longer range sequence context such as generation of synthetic severe acute respiratory syndrome coronavirus 2 (SARS-CoV-2) sequences. GenSLMs can help predict evolutionary trajectories for viral genomes. The models 112 can include a MORL algorithm. The models 112 can be fine-tuned passed on the plurality of reward metrics, e.g., rewards. The models 112 can generate one or more protein sequence elements based on an initial protein sequence structure.

The system 100 can include training data 108. The system 100 can retrieve the training data 108 from and/or store the training data 108 in one or more data sources that can be maintained by the system 100 and/or be remote from the system 100.

The training data 108 can include or be retrieved from one or more data sources, such as data sources that include data structures that represent protein sequences. For example, the data structure can include a plurality of sequence elements, each sequence element corresponding to at least one of a codon or an amino acid of a protein sequence. The training data 108 can include diverse protein/gene sequence datasets. These datasets can include a variety of protein and gene sequences. The training data 108 can include natural malate dehydrogenase (MDH) sequences which can include approximately 36,000 sequences. The training data 108 can include data of a genomics and/or bioinformatics-based dataset, such as the pathosystems resource integration center (PATRIC) dataset.

The training data 108 can include input features and corresponding target outputs. The input features can include protein sequences, organisms, and genome sequences. Target outputs can include data associated to the input features such as sequence length, sequence folding, and stability. The training data 108 can be preprocessed, e.g., cleaning data to remove missing values. The training data 108 can be included in or provided to the model trainer 106 for the model trainer 106 to use to train models. The models 112 can be models that have been trained by the model trainer 106 on the training data 108.

### Reinforcement Model-Based Protein Sequence Generation and Evaluation

Referring further to FIG. 1, the system 100 can include the sequence generator 110. The sequence generator 110 can generate protein sequences, such as to predict one or more sequence elements of a protein sequence (e.g., given at least an initial sequence element of the protein sequence). The sequence generator 110 can generate genome sequences. For example, the sequence generator 110 can be or include one or more models 112, which can generate protein sequences and/or genome sequences, such as in response to receive an indication of an initial sequence (e.g., one or more codons to provide a starting point for sequence generation) and/or a characteristic for the sequence to be generated (e.g., a target for the sequence).

The sequence generator 110 can generate novel gene sequences that can be constrained by evolutionary conservation at a protein sequence level, e.g., at the amino-acid level by translating codons to corresponding amino acids. For example, by implementing evolutionary conservation, the sequence generator 110 can retain combinations of amino acids that were rewarded by the model 112. The sequence generator 110 can transfer knowledge and/or policies from previous runs, e.g., iterations of the model 112 to generate new sequences. The sequence generator 110 can transfer learning from one objective to another objective. The sequence generator 110 can transfer learning from one iteration of the model 112 to a following iteration of the model 112. For example, the sequence generator 110 can use information learned from one objective and apply the information to another objective.

The sequence generator 110 can generate protein sequences based on rewards determined from multiscale molecular dynamic (MD) simulations and experimental data. For example, the sequence generator 110 can constrain protein generation to amino acid combinations with positive feedback, e.g., rewards. The sequence generator 110 can apply limits and generate sequences based off of rewards from the model 112. The sequence generator 110 can apply rewards and results from the MD simulations to generate sequences. The sequence generator 110 can apply experimental data to generate sequences. For example, the sequence generator 110 can remove sequences that were given penalties from the MD simulations or the model 112. For example, the sequence generator 110 can utilize the experimental data to remove protein sequences and prioritize protein sequences. The sequence generator 110 can continually constrain and/or refine itself based off of feedback from the model 112 to generate better protein sequences based off of the objective, e.g., more stable proteins.

The system 100 can include the sequence scorer 114. The sequence scorer 114 can determine one or more scores for any one or more protein sequences generated by the sequence generator 110. The sequence scorer 114 can determine scores for protein sequences received from the sequence generator 110. The sequence scorer 114 can run scoring tasks on sequences generated by the sequence generator 110. The processor 102 can cause the sequence scorer 114 to score sequences generated by the sequence generator 110.

The sequence scorer 114 can score folded protein sequences, and can use similarity (e.g., a metric of similarity) to compare the folded protein sequences to existing protein sequences. The sequence scorer 114 can rank protein sequences based on the similarity of the generated protein sequence to existing, e.g., natural, protein sequences. The sequence scorer 114 can determine the similarity by performing a semantic similarity search. The semantic similarity search can refer to a computational process of retrieving from a database item that are relevant to a meaning of a query. The database can be a database that includes existing genome and protein sequences. The query can be the generated protein sequence.

The sequence scorer 114 can map semantic information about the generated sequences, e.g., guanine-cytosine (GC) content and molecular weight, into a latent space of dense vector representations. The latent space can be a representation that captures characteristics or features of input data with reduced dimensionality. Such latent spaces have become attractive mediums for efficient semantic similarity search due to hardware-accelerated vectorized operations. Semantic similarity search can enable encoding 36,622 MDH sequences with a version of a 25M parameter GenSLM, e.g. 25 million parameter language model, that has been fine-tuned on MDH sequences and store the resulting 512-dimensional embeddings in a vector database. The vector database can be implemented as a Faiss index. The Faiss index can support efficient indexing algorithms ranging from Inverted File Systems (IVF) to Hidden Navigable Small Worlds (HNSW) boosted by GPU acceleration for handling trillion-scale databases. A flat index can be employed in which a distance metric between two embeddings e₁, e₂∈ [symbol] can be defined as the inner product [symbol] e₁, e₂ [symbol] .

The Faiss index can be leveraged to implement a novelty reward in the model 112. Each batch S of a generated or synthetic protein sequence can be encoded. For each generated or synthetic protein sequence s E S a novelty score can be computed by:

$$Nov . ( s ) = \frac{1}{10} {\sum }_{n = 1}^{10} 〈 {e}_{s} , {e}_{n} 〉$$

where eₛ is the embedding of s and e₁, . . . e₁₀ are the 10 nearest neighbors of eₛ in the latent space. The novelty reward can incentive the sequence generator 110 to produce protein sequences whose latent embeddings are distant from pre-existing protein sequences. For example, the novelty reward can incentive the sequence generator 110 to produce MDH variants whose latent embeddings are distant from those of the pre-existing MDH variants. Distance in the latent space can correlate with a semantic distance in physicochemical properties. The novelty reward can drive the model 112 to produce performant and novel protein sequences. For example, performant and novel MDH variants. Performant protein sequences can refer to sequences that exhibit desirable characteristics or properties.

To accelerate calculations of the novelty reward, the sequence encodings and Faiss nearest-neighbor search can be distributed across multiple graphics processing units (GPU). The GPU can be coupled to the memory 104 and aid in computing. For example, for a batch of 1,024 sequences, an average time (e.g., real-world elapsed time) of four seconds can be achieved to compute the novelty reward. Semantic similarity search by the sequence scorer 114 can aid the system 100 in ranking and determining which generated protein sequences should be evaluated. The sequence scorer 114 can include a score storer that collects results of the scoring tasks or results of the sequence scorer 114 and store them in a database.

The plurality of scoring functions used by the sequence scorer 114 can comprise a similarity function based on a database of example sequence data, a folding function, and a stability function. The sequence scorer 114 can score using the plurality of stability functions responsive to an output of at least one of the similarity function or the folding function satisfying a corresponding threshold.

The sequence generator 110 can submit tasks to generate more sequences in response to all previous protein sequences having been scored, e.g., by the sequence scorer 114, or a new RL model can be available. The sequence generator 110 can launch scoring tasks for each new protein sequence. The processor 102 can initiate the sequence generator 110 to generate more protein sequences in response to all previous protein sequences having been scored. The sequence generator 110 can indicate to the processor 102 to launch scoring tasks for each new protein sequence generated.

The system 100 can include a stability evaluator 116. The stability evaluator 116 can receive scored sequences form the sequence scorer 114, and evaluate a stability of each of the scored sequences. The stability evaluator 116 can use atomistic molecular dynamic (MD) simulations to verify whether the scored sequences can form a stable protein fold and provide an extrinsic reward function that rewards the RL with a protein stability related measure. MD simulations can be computational methods of simulating a motion of molecules, atoms, and functional groups. 3D structures of the generated sequences can be predicted using a folding function, e.g., ESMFold, and relaxed using MD simulations with explicit solvent, e.g., water box and counter ions to neutralize the generated sequences. The folding function can be a mathematical or computational method of predicting a 3D structure of the generated protein sequence. The folding function and can include per-residue log-likelihood-distance transformation (plddt) scores. Plddt scores can provide an estimation of confidence or reliability of the generated sequence. ESMFold can be used for end-to-end folding, e.g., predicting the 3D structure of the generated protein sequence from end-to-end of the protein sequence. Molecular topology, including all atomic interactions, can be modeled using MD simulations, e.g., Ambertools using the Amberl4SB force field and tip3p water model. Molecular topology can include spatial arrangement, connectivity, and other relationships and characteristics between atoms and functional groups of molecules. MD simulations can be carried out with OpenMM in isothermal-isobaric (NPT) ensemble at 310 K and 1 bar for a total of 10 ns for each generated sequence. A timestep of 2 fs with Langevin integrator with 10 Å cut-off for non-bonded interactions can be used for the MD simulations. The Langevin integrator can be an algorithm to model dynamics of particles in a system. Long-distance interaction of molecules or functional groups or atoms in the MD simulations can be corrected with Particle Mesh Ewald (PME) methods. Reward computation at an end of the MD simulation can be a measure of the root mean squared fluctuations (RMSFs) for the Ca atoms in the simulation.

For example, given approximately 90 crystal structures available for MDH sequences the average RMSF across experimentally determined crystal structures can be 2.3 Å. 2.3 Å can imply that any sequences >2.3 Å may not be suitable for further characterization, e.g., the activity evaluator 118. The system 100 can restrict the stability evaluator 116 to allow more stable structures, e.g., <2.3 Å. The model 112 can be rewarded by a result of a structure being stable from the stability evaluator 116. The processor 102 can initiate the stability evaluator 116 to evaluate the stability of the sequence.

The system 100 can include the activity evaluator 118. The activity evaluator 118 can receive the stability evaluated and scored sequences from the stability evaluator 116 and evaluate an activity of each sequence. The activity can be enzymatic activity. The activity can be estimated by empirical valence bond (EVB) and umbrella sampling. For example, EVB and umbrella sampling can be used to estimate the enzymatic activity of generated MDH designs/sequences. EVB can be a computational method that includes the valence bond (VB) theory, e.g., electronic structure of molecules. Umbrella sampling can be a computational method used in MD simulations to sample areas of interest in the generated protein sequences. EVB and umbrella sampling can be implemented using the MD simulation, e.g., OpenMM, on GPU. The implementation on GPU can complement approaches implemented on CPU platforms such as Amber, Polaris, and Q6.

For example, during the MDH process, both a proton (H+) and a hydride ion (H−) can transfer from malate to NAD+, reducing the proton and the hydride ion to NADH. Since the hydride transfer can be considered a rate-limiting step, the hydride transfer can be modeled with an EVB approach implemented in the MD simulation. The rate-limiting step can be the step that determines a rate of reactions. The rate-limiting step can include the step with the greatest activation energy relative to other steps. The rate-limiting step can include the step that is a transition state with the highest free energy relative to other steps.

For example, two molecular systems were built to represent both the reactant (malate/NAD+) and product (oxaloacetate/NADH) states of the reaction. Chemical bonds involving the transferring hydride can be described with a Morse potential, instead of the standard harmonic potential. The Morse potential can be a mathematical function that describes potential energy between two atoms or molecules as a function of a separation distance between the two atoms or molecules. The hydride was bonded with malate C2 atom in a reactant state and transferred to the NADH nicotinamide ring in a reaction product. A difference between bond lengths of the two hydride bonds was used as a reaction coordinate (RC). The RC can be a representation of a progress of a reaction. In the umbrella sampling process, a harmonic potential, with 5000 kJ mol-1 Å-2 was added to fix the RC at each sampling point from −0.6 to 0.6 Å with 0.05 Å increment. Harmonic potential can be a mathematical model to describe potential energy of a system. The simulations resulted in a total of 26 simulations with 13 sampling points for each state. The simulations were run for 1 ns with similar conditions described above. A RC reporter was used to record the RC every 1 ps. The simulation results were then processed using the Weighted Histogram Analysis Method (WHAM) for the potential mean force of the hydride transfer.

The activity evaluator 118 can include EVB simulation results which can include a free energy barrier height of the reactant and product state. The EVB simulation results can aid in evaluation of a turnover rate (k_{cat}) for the enzyme using the Arrhenius equation. k_{cat} can be used by the activity evaluator 118 to evaluate the activity of the generated protein sequence. An output of the activity evaluator 118 can be used as an award to drive the models 112, e.g., the RL generative modeling. A balance between the activity evaluator 118 and the stability evaluator 116 can be made by explicitly pruning, removing, or deleting a number of equilibrium MD simulations, e.g., run by the stability evaluator 116, while maintaining the EVB-MD simulations, e.g., run by the activity evaluator 118, to consider only the most promising (e.g., viable) generated sequences. For example, the activity evaluator 118 can include at least one threshold and compare each of the generated sequences to the threshold. The threshold can indicate a viability of the generated sequence, and the activity evaluator 118 can input the generated sequences greater than the threshold to the simulations to reduce a computation load and solution search space. The processor 102 can initiate the activity evaluator 118 to evaluate the activity of the generated sequence.

The system 100 can output one or more selected protein sequences of a subset of selected protein sequences. The selected protein sequences can be sequences that have passed through, e.g., received rewards (e.g., received rewards that satisfy corresponding metrics or thresholds), from the sequence scorer 114, the stability evaluator 116, and the activity evaluator 118. The selected protein sequences can be sequences that satisfy the objective of the model 112. The selected protein sequences can be output into a database, an array, a list, and/or table. The selected protein sequences can be run through simulations to generate a 3D model. The selected protein sequences can be fabricated and tested for experimental data. The selected protein sequences can be used to identify new and emergent variants of pandemic-causing viruses, e.g., SARS-CoV-2. The selected protein sequences can represent evolutionary dynamics of pandemic-causing viruses.

FIG. 2 depicts a system 200 for MORL controlled generation of genome/protein design. The system 200 can be incorporated in the system 100. The system 200 can incorporate successively complex rewards, e.g., rewards from the activity evaluator 118, determined from evolutionary conservation, MD simulations, and experimental observations. The system 200 can enable constraining generative modeling to respect design parameters in synthetic biology applications. The system 200 can include and/or execute one or more components and/or operations such as a foundation model 202 (e.g., trained on PATRIC sequences), a process 204 to finetune model on protein target(s), the model 112, a first agent RL policy 208, a second agent RL policy 210, a k agent RL policy 212, a first reward model 214, a second reward model 216, a k reward model 218, a sequence 220, a structure 224, kinetics 226, energetics 228, a first reward 230, a second reward 232, a m reward 234, and a multi-objective loss function 236. In some implementations, various such components of the system 200 can be combined, not included in the system 200, and/or managed by components separate from the system 200.

The foundation model 202 can have varying parameter sizes from 25 million to 25 billion parameters. The foundation model 202 can be trained on genome and protein sequences. The foundation model 202 can be trained on 110 million, diverse PATRIC sequences. The foundation model 202 can be a language model. The foundation model 202 can be a GenSLM. The foundation model 202 can be a protein-scale language model (ESM). The foundation model 202 can follow a standard casual modeling training scheme, e.g., including selecting variables and estimating parameters. Architecture, e.g., design, of the foundation model 202 can follow, e.g., model, a natural language processing (NLP) model. The foundation model 202 can be an NLP model. For example, the foundation model 202 can follow a GPT-NeoX style of decoder-only transformer with Rotary Position Embedding (RoPE) which can replace a standard learned positional embedding layer. GPT-NeoX can be an NLP model while RoPE can be a technique that can incorporate positional information into input of the NLP. The standard learned positional embedding layer can learn position embeddings of an input sequence. Position embeddings can encode a position of tokens within a sequence, e.g., a position of a codon within a protein sequence.

The foundation model 202 following the NLP can allow for adaptation of a maximum sequence length (e.g., from 2,048 sequence elements) and can allow for more efficient training under data regimes that do not use large context lengths or increased context for datasets with longer sequences. Genomic sequence inputs for the foundation model 202 can be tokenized using a codon-level tokenizer which can split genomes into blocks of 3 nucleic acids comprising adenosine (A), cytosine (C), guanosine (G), and thymidine (T). Tokenizing the foundation model 202 can result in 64 unique textual tokens. The foundation model 202 can include utility tokens comprising padding, mask, unknown, cls, and separator. The utility token can be a tool, technique, method, process, system to enhance processing, analysis, or understanding of the foundation model 202. The mask utility token can enable the foundation model 202 to perform masked language modeling (MLM) or other mask prediction tasks. The unknown utility token can represent tokens not present in a vocabulary of the foundation model 202. The cls utility token can be used to classify and process sequences. The separator utility token can separate two sequences. The padding utility token can ensure that all sequences in a batch have a same length. The 64 unique textual tokens and the utility tokens can provide a vocabulary size of 69, for example.

A dataset for the foundation model 202 can include sequences from PATRIC (e.g., 110 million sequences from PATRIC). A dataset for the foundation model 202 can comprise 110 million sequences from The Bacterial and Viral Bioinformatics Resource Center (BV-BRC) database. BV-BRC can allow for an aggregation of protein sequences of similar function across genera. The aggregation of protein sequences of similar function across genera can be leveraged to collect more than 10,000 unique protein function families (PGfams) which can combine to form the 110 million gene sequences used for the pre-training of foundation model 202.

The foundation model 202 can include an ESM2 model. The ESM2 model can represent a similar scale of models trained on amino acid language instead of gene sequences. ESM2 can provide models with a number of trainable parameters ranging from 8 million to 15 billion trainable parameters trained on 65 million unique protein sequences taken from UniRef50 and UniRef90 datasets. The UniRef50 and UniRef90 datasets can be databases provided by the UniProt Consortium. Architecturally, ESM2 models can follow Bidrectional Encoder Representations (BERT)-style transformers and use a masked language training scheme. BERT can pretrain transformer-based models.

The process 204 can include finetuning the foundation model 202 based on targets. For example, the foundation model 202 can be tuned by training the foundation model 202 on gene sequences (e.g., 110 million prokaryotic gene sequences for a SARS-CoV-2-specific model on 1.5 million genomes). The process 204 can be finetuned with explicit feedback, e.g., from the stability evaluator 116 and experiments. The process 204 can be finetuned by RL models applied to the foundation model 202 which can be a pre-trained language model for specific tasks. The process 204 can be tuned to focus on a specific objective, e.g., protein sequences with a minimum stability threshold. The process 204 can be further depicted in FIG. 3.

The model 112 can be a model that has been pre-trained on sequences and finetuned. The model 112 can be a MORL model, a GenSLM, an RL model, or other algorithm. For example, the model 112 can be the GPT-NeoX for PLM and the GPT-NeoX variant employed in GenSLM that were trained on diverse protein/gene sequence datasets respectively.

The system 200 can include the first agent RL policy 208 which can define key actions. The key action can include “update the model used for generation after training completes, then start training again with the latest experimental data.” The first agent RL policy 208 can be trained to optimize a specific objective. The specific objective can be proteins with a specific structure, e.g., tertiary. The first agent RL policy 208 can be π=ρ, where p is a pre-trained LLM, e.g., either genome-scale language model/GenSLM or a protein language model/PLM. Unlike other applications where human feedback is used, ρ can be obtained from pre-trained GenSLMs or PLMs. The first agent RL policy 208 can be fine-tuned using RL to perform the gene/protein sequence-specific generation task informed by a multi-objective reward model. The second agent RL policy 210 and the k agent RL policy 212 can replicate the first agent RL policy 208 with varying key actions or objectives. For example, an objective of the first agent RL policy 208 can be folding while the second agent RL policy 210 can be stability of the generated sequences. The first agent RL policy 208, the second agent RL policy 210, and the k agent RL policy 212 can be referred to as an agent RL policy and can be further depicted in FIG. 3. The k agent RL policy 212 can represent at least a third or more of the agent RL policy. The agent RL policy can be a plurality of agents, each agent of the plurality of agents associated with a different reward metric, e.g., reward model, of the plurality of reward metrics than each other agent of the plurality of agents. A plurality of the agent RL policy can generate for each protein sequence a plurality of examples of protein sequences, a reward score for an objective function, the reward score generated based on a different metric for each agent of the plurality of agent RL policy. The system 200 can include evaluating the objective function using each reward score to generate an output of the objective function and updating the model 112 based on the output. The metric of the first agent RL policy 208 can correspond to a structure of the protein sequence and the metric of the second agent RL policy 210 can correspond to kinetics of the protein sequence. The model 112 can include the agent RL policies.

The system 200 can include the first reward model 214 which can be associated with the first agent RL policy 208 and can be associated with a metric or objective. The first reward model 214 can reward the first agent RL policy 208 based on the metric or objective. For example, the first reward model 214 can be for folding and can be formulated with the plddt score averaged across residues. Sequences with average plddt score >0.7 can be rewarded and the rest can be penalized. For example, the first reward model 214 can be for MD using the RMSF value averaged across residues to be below 2.5 A. For example, the first reward model 214 can be for EVB runs and can be formulated using the height of the reaction profile averaged across the reaction coordinate. In response to an average height of a reaction profile being below the corresponding value for a wild type MDH, the corresponding candidate can be rewarded. For example, the first reward model 214 can constrain diversity of generation via evolutionary rewards or semantic similarity. The second reward model 216 and the k reward model 218 can replicate the first reward model 214 but have different objectives, e.g., the first reward model 214 rewards sequence length and the k reward model 218 rewards kinetics of a sequence. For example, the first reward model 214 can reward folding while the second reward model 216 can reward the average height of the reaction profile in an EVB run. The first reward model 214, the second reward model 216, and the k reward model 218 can herein be referred to as reward models and can be further depicted in FIG. 3. The k reward model 218 can represent at least a third or more reward models. The reward models can include a classifier reward model using the foundation model 202, e.g., ESM2 pre-trained model having 35 million parameters with an added sequence classification head. The model 112 can include the reward models.

The system 200 can include the sequence 220, the structure 224, the kinetics 226, and the energetics 228. The sequence 220, the structure 224, the kinetics 226, and the energetics 228 can herein be referred to as objectives and can be examples of objectives of the first reward model 214, the second reward model 216, and the k reward model 218. The model 112 can incorporate successively complex rewards based on the objectives. Rewards can be based on successful achievement and/or accomplishment of the objective of the model 112. The model 112 can incorporate rewards from evolutionary conservation, MD simulations, and experimental observations of the generated sequences. For example, kinetics 226 can be associated, determined by MD simulations. Evolutionary conservation metrics can be a percentage of amino acids in a sequence, similarity, a conservation score, and any factor that can quantify a degree of conservation of a sequence across different species. For example, evolutionary conservation metric can be associated, determined by a reward model involving the sequence 220 and the structure 224. Experimental observations can be determined by feedback of the first reward model 214, the second reward model 216, and the k reward model 218. The reward models can use, but not limited to, the sequence 220, the structure 224, the kinetics 226, and the energetics 228 as rewards, objectives, and/or targets. The model 112 can include the objectives.

The system 200 can include the first reward 230, the second reward 232, and the m reward 234 and can herein be referred to as rewards. The first reward 230 can be associated with and be a result of the first reward model 214. The second reward 232 can be associated with and be a result of the second reward model 216. The m reward 234 can be associated with and be a result of the k reward model 218. Rewards can be based on a degree of achievement or success of the model 112 of the objective provided by the agent RL policies. The reward model can generate the rewards. The rewards can be used to finetune the model 112. The rewards can be drawn from evolutionary conservation metrics. The rewards can be drawn from multi-scale molecular simulations such as an all-atom molecular dynamic (AAMD) and EVB approaches estimate catalytic efficiency kcat. The rewards can be drawn from experimental data. Experimental data can be data from lab experiments performed by humans. The rewards can be intrinsic or extrinsic. Rewards can be used by the system 200 to adjust the model 112 output. For example, the rewards can be a classifier reward, an evolutionary reward, or a semantic similarity reward. Rewards can be further depicted in FIG. 3. The m reward 234 can represent at least a third or more reward. The model 112 can include the rewards.

The rewards can be evolutionary rewards. Evolutionary rewards can be associated with an ability protein to fold, perform biochemical activities, and adapt and can be determined by its primary sequence which can dictate interactions between amino acid residues. Distinct relationships between protein sequence and functions can be optimized via evolution. Statistical Coupling Analysis (SCA) can be one technique that can be used to uncover co-evolutionary patterns from the primary sequence alone by using a large group of related sequences. Related sequences can be identified using multiple sequence alignments (MSAs). MSAs can be computed using a multiple sequence alignment program. The multiple sequence alignment program can be an MAFFT package. The MAFFT package can offer a relative advantage of speed and flexibility over accuracy compared to other multiple sequence alignment programs.

The system 200 can include the multi-objective loss function 236. The multi-objective loss function 236 can incorporate results from the first agent RL policy 208, the second agent RL policy 210, and the k agent RL policy 212. The multi-objective loss function 236 can incorporate the first reward 230, the second reward 232, and the m reward 234 to improve a performance, results, accuracy of the model 112. The multi-objective loss function 236 can provide feedback on the model 112 performance. The multi-objective loss function 236 can also be a cost or objective function. The multi-objective loss function 236 can balance, capture, weigh results of the agent RL policies. The multi-objective loss function 236 can optimize multiple objectives or criteria or agent RL policies simultaneously.

FIG. 3 depicts a system 300 that can execute an instance (e.g., a single instance, a loop) of fine-tuning an RL model. The system 300 can be a singular instance of the agent RL policy depicted in FIG. 2. The system 300 can be designed, configured, adapted to optimize a specific objective.

The system 300 can involve 2 models. The 2 models can be the foundation model 202 which can be a pre-trained LLM and a reference LLM that can be used to determine a policy gradient loss via Kullback-Leibler (KL)-divergence. The policy gradient loss can be a loss function designed, configured, adapted to find policy parameters to maximize an expected return, e.g., functional designed sequences. The KL-divergence can be a method to quantify a difference between probability distributions. The system 300 can double a computational and memory range for model fine-tuning runs compared to traditional pre-training of LLMs. The system 300 can include a seed model 302, a policy module 304, a sequence generator 316, a reward model 318, rewards 320, and RL loss 322. The policy module 304 can include a model/policy 306, a reference model/policy 308, a V_{f}(r) 310, a KL division (KL div) 312, and a proximal policy optimization (PPO) Loss 314.

The system 300 can include the seed model 302. The seed model 302 can be a pre-trained model. The seed model 302 can be a pre-trained LLM. The seed model 302 can be the GenSLM. The seed model 302 can be the PLM. The seed model 302 can be trained to predict the next token(s) in the sequence. Tokens can refer to either a 3-letter nucleotide combination referred to as a codon, or an amino acid residue. The codons can encode a total vocabulary of 4³=64 tokens mapped onto 20 naturally occurring amino acid residues constituting respective vocabulary sizes of the seed model 302. The seed model 302 can be prompted with a start token or with a first few tokens of a sequence.

The policy module 304 can be a module that initializes a policy. The policy module 304 can correspond to the first agent RL policy 208, the second agent RL policy 210, and the k agent RL policy 212. The policy can be π=ρ, where ρ is a seed model 302. The initialized policy π can be fine-tuned using RL to perform a gene/protein sequence-specific generation task informed by a multi-objective reward model. The policy can be the model/policy 306.

The model/policy 306 can be policy π(at |st). The model/policy 306 can predict a next token conditional on the previous tokens and can be seeded by the seed model 302 that defines a probability distribution over a sequence of tokens Vₜ via

$$ρ ( {a}_{0} ⋯ {a}_{t - 1} ) = {\prod }_{0 \leq k \leq t} ρ ( {a}_{t} \vert {a}_{0} ⋯ {a}_{t - 1} ) .$$

The reference model/policy 308 can be a previous iteration of the system 300 and can be used to ensure the model/policy 306 being run does not substantially differ from the reference model/policy 308. The V_{f}(r) 310 can be a value function and can estimate expected future rewards. The KL div 312 can be a measure of a divergence between two probability distributions. The two probability distributions can be derived from the model/policy 306 and the reference model/policy 308. The KL div 312 can determine if the model/policy 306 begins to drastically differ from the reference model/policy 308. The KL div 312 can use a log function to determine a difference between the model/policy 306 and the reference model/policy 308. The PPO Loss 314 can be a policy gradient method to train the model/policy 306. The PPO Loss 314 can be designed, configured, adapted to optimize policy parameters by maximizing rewards while maintaining, stabilizing an efficient learning of the model/policy 306.

The policy module 304 can include a linear layer with randomized initialized weights that can be added to a casual language modeling head of the seed model 302. The linear layer can act as a value head and can approximate V_{f}(r) 310 for the PPO Loss 314.

The model/policy 306 can be initialized by the reference model/policy 308, the V_{f}(r) 310, the KL div 312, and the PPO Loss 314. The policy module 304 can maintain an instance, e.g., a run, training, of the seed model 302 as the reference model/policy 308. The policy module 304 can include a KL constraint to ensure that the model/policy 306 while getting fine-tuned does not diverge drastically from the reference model/policy 308. The policy module 304 can maximize expected cumulative rewards obtained by the first agent RL policy 208, the second agent RL policy 210, and/or the k agent RL policy 212.

The sequence generator 316 can generate protein or genome sequences based on results of the policy module 304. The reward model 318 can be, but not limited to, a folding reward model, an MD reward model, an EVB reward model, an evolutionary reward, or a semantic similarity reward model The reward model 318 can correspond to the first reward model 214, the second reward model 216, and the k reward model 218.

The reward model 318 can include a “fitness” measure for the generated sequences. The “fitness” measure can be obtained from both sequence-specific factors (intrinsic) and experimental data (extrinsic). The experimental data can include measures of catalytic efficiency (for an enzyme). The “fitness” measure can be a multi-objective reward of the reward model 318.

For example, one of the objectives of the reward model 318 can be to ensure that a GC content and a length of the protein's primary amino-acid sequence of the generated sequences are within an expected range of values, e.g., target output. The GC content and the length can be collectively captured by a sequence classifier as a reward output by the reward model 318.

The rewards 320 can correspond to the first reward 230, the second reward 232, and the m reward 234. The rewards 320 can be combined using a scalarization approach by assigning appropriate weight to each reward as shown:

$${r}_{sequence} = {\sum }_{a} {w}_{a} {r}_{a}$$

with α∈(PF, plddt, MD-RMSF, EVB-Height), where PF denotes a protein family or a subfamily.

The reward model 318 can provide, give, determine rewards 320 based on the sequences generated by the sequence generator 316. The seed model 302 can be prompted with a start token or with a first few tokens of a sequence and a completed sequence can be expected to satisfy the rewards 320.

The model/policy 306, e.g., w, can be finetuned to optimize the reward model 318. To keep w from moving too far from p, a penalty can be added with expectation and the model/policy 306 can be trained with the following objective:

$$obj . ( ϕ ) = {E}_{( x , y ) \sim {D}_{ϕ}^{{π}^{RL}}} [ r ( x , y ) - β log ( \frac{{π}_{ϕ}^{RL} ( y \vert x )}{π ( y \vert x )} ) ]$$

The above objective can be a part of, incorporated and/or included in the RL Loss 322. The RL Loss 322 can be a loss function used to train the policy module 304. The RL Loss 322 can train the policy module 304 via back propagation. Back propagation can be used to adjust weights used by the policy module 304.

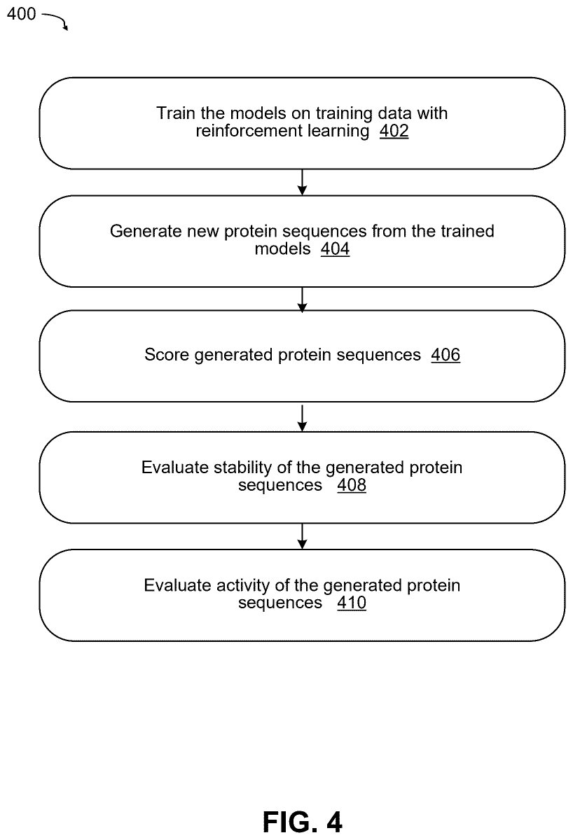

FIG. 4 depicts a method 400 for the multi-objective reinforcement learning approach for protein design incorporating experimental data. The method 400 can be performed using various systems described herein. Various steps in the method 400 may be repeated, omitted, performed in various orders, or otherwise modified. Various steps in the method 400 may be run concurrently, in parallel, or individually.

At 402, models are trained on training data, e.g., training data 108, with reinforcement learning. The models can include large language models (LLMs) with reinforcement learning (RL) to generate better protein sequences.

At 404, new protein sequences are generated from the trained models. The new protein sequences are generated based on the latest weights of the model, e.g., the LLM. The system 100 can generate, by one or more processors 102 using a machine model, e.g., the model 112, based on an initial protein sequence data structure, a plurality of protein sequences, the machine learning model, e.g., the model 112 configured based on RL from a plurality of reward metrics, e.g., rewards, including at least one reward metric associated with experimental data regarding example sequence data. 404 can include generating a plurality of protein data sequences comprising generating a plurality of protein sequence data elements, for each protein sequence data sequence of the plurality of protein data sequences, wherein each protein sequence data element of the plurality of protein sequences data elements represents at least one of a codon or a protein residue. The plurality of reward metrics can comprise at least one evolutionary conservation metric and at least one molecular simulation metric.

At 406, generated protein sequences can be scored. Scoring the generated protein sequences can facilitate determining which protein sequences are most promising to evaluate further. The system 100 can score, by the one or more processors 102, using a plurality of scoring functions, e.g., by the sequence scorer 114. The plurality of scoring functions used can comprise at least one function based on at least one of the GC content or molecular weight of each protein sequence of the plurality of protein sequences.

At 408, the stability of the generated protein sequences can be evaluated. The stability of the protein sequences can be evaluated using molecular dynamics.

At 410, the activity of the generated protein sequences can be evaluated. The activity can be enzymatic activity. The enzymatic activity can be evaluated by EVB and umbrella sampling.

### Computing Task Management for Reinforcement Model Training and Protein Sequence Generation

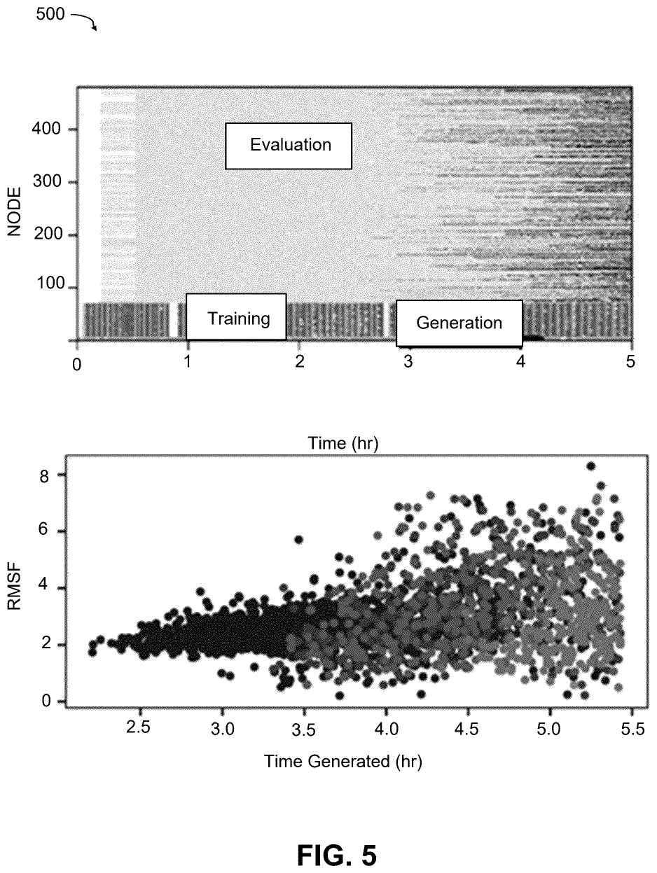

Referring further to FIGS. 1-4 (and as described, for example, with reference to FIG. 5), the system 100 can asynchronously perform, by the one or more processors 102 in parallel using a plurality of parallel computing resources, at least one of the generating of the plurality of protein sequences or the scoring of the plurality of protein sequences. Parallel computing resources can include hardware and software that can enable simultaneous and/or asynchronous execution of multiple tasks or computations.

As noted above, various systems described herein can implement one or more computer operations and/or combinations thereof as tasks. The tasks can include generating the protein sequence, scoring the sequence, folding the sequence, and evaluating the sequence (e.g., based on operation of components such as the sequence generator 110, sequence scorer 114, stability evaluator 116, and/or activity evaluator 118). The RL training tasks can include prediction or simulation tasks.

The model trainer 106 can submit RL training tasks onto multiple computer nodes, which can be individual computing units. For example, the model trainer 106 can submit one or more RL training tasks onto one or more computer nodes. The model trainer 106 can submit the model 112 with the objective of tertiary structure protein sequences onto one computer node, and another model 112 with the objective of a minimum stability onto another computer node. The model trainer 106 can submit generating the protein sequence, scoring the sequence, folding the sequence, and evaluating the sequence onto one or more computer nodes. The processor 102 can initialize the sequence generator 110, the sequence scorer 114, the stability evaluator 116, and the activity evaluator 118 onto one or more computer nodes.

The model trainer 106 can launch new trained RLs, e.g., models 112, as soon as previous models complete. The model trainer 106 can launch new trained RLs as soon as previous models output protein sequences. The model 112 can be sequentially and/or concurrently finetuned by feedback the model trainer 106 receives from completed model 112 runs and/or iterations. The model 112 can be finetuned by feedback the model trainer 106 receives from one or more computer nodes. One or more of the model 112 can be finetuned by the model trainer 106 based on feedback received from one or more computer nodes.

The model trainer 106, the sequence generator 110, the sequence scorer 114, the stability evaluator 116, and the activity evaluator 118 can run concurrently, simultaneously, in parallel, individually, sequentially and can create a workflow that can use each time of computation effectively at scale. One or more of the system 100 can run concurrently, simultaneously, in parallel, individually, and/or sequentially. A single run of the system 100 can deploy tasks that utilize less than a single GPU up to hundreds of GPUs and tasks which can produce kBs of data to 100s of MBs. The single run of the system 100 can use 480 nodes and can deploy 1648 parallel scripting workers that each perform one RMSF computation or three runs from an EVB computations concurrently. For example, Nvidia's Multiprocessing Service yields a 2× throughput increase through oversubscription. The parallel scripting workers can be Parsl. Tasks can be dedicated on 32 nodes using deep learning optimization library and a collective communications library backend. The deep learning optimization library can be DeepSpeed and the collective communications library can be Nvidia Collective Communication Library (NCCL). Remaining nodes can alternate between generating new protein or genome sequences, evaluating novelty of the sequences with the Faiss index, and folding the sequences.

For example, FIG. 5 depicts a plurality of tasks being run on a plurality of computer nodes simultaneously. A time to run the plurality of tasks on the plurality of computer nodes can vary. For example, training tasks can take a shorter amount of time than generations tasks. FIG. 5 is further described herein.

### Performance Evaluation

The system 100, e.g., the MORL approach for protein/gene design incorporating experimental data, can be demonstrated by designing MDH, an enzyme within the citric acid cycle, which reversibly catalyzes the oxidation of malate to oxaloacetate via the reduction of nicotinamide adenine dinucleotide (NAD), using genome-scale language models, and lysozyme (LYZ), an antimicrobial enzyme, using protein language models. The approach can be broadly applicable to other enzymes, protein-protein complexes, and pathways.

For MDH sequences, the classifier model, e.g., the reward model, as a binary classifier was finetuned with sequences having GC content between 45-60 as class 1 and the rest of the sequences with GC<45 and GC>60 as class 0. For Lysozymes, the classifier was trained as a multi-label classifier using 3 labels for the 3 subfamilies.

For Lysozymes, the system 100 was applied on a PLM model pre-trained on 3 sub-families of lysozymes (PF.) with amino acid vocabulary. For MDH sequences, the approach was applied on GenSLM models fine-tuned on 36,000 MDH sequences. The objective in the Lysozyme example, was to demonstrate that the system 100 can align the PLM to generate more of a subfamily of interest over the other families compared to baseline generation. In case of MDH, the equivalent objective was to align the GenSLM to generate more sequences in with GC content falling within an interval of 45-60. These objectives are well suited for a classifier type of reward model.

For training the policy π, the PPO2 version of Proximal Policy Optimization was chosen. The GenSLM model was first pre-trained on the PATRIC dataset and then fine-tuned on the natural MDH sequences with approximately 36,000 sequences. The fine-tuned model was used as the starting model for the subsequent policy training for MDH sequences.

Fine-tuning was performed for 50 epochs and was stopped in response to the perplexity of the fine-tuned model reached approximately 3. The policy was then initialized on the fine-tuned model. A batch size of 64 sequences was used since the limiting step in the reward feedback was the folding computation which takes approximately 1 minute per sequence for the generated MDH sequences with sequence lengths in the range of 250 to 350 codons. Further, during the training of the language policy model the model perplexity was monitored to ensure that the model does not diverge from its initial perplexity. Keeping the perplexity values within an expected threshold can ensure that the generated sequences respect the sequence grammar of the protein/gene family of interest by maintaining the β KL penalty term in obj.

$$( ϕ ) = {E}_{( x , y ) \sim {D}_{ϕ}^{{π}^{RL}}} [ r ( x , y ) - β log ( \frac{{π}_{ϕ}^{RL} ( y \vert x )}{π ( y \vert x )} ) ] to be 0.1 .$$

MSAs were computed using the MAFFT package. MAFFT offers the relative advantage of speed and flexibility over accuracy compared to other techniques such as BLAST. For the sequences considered here, including malate dehydrogenase (MDH) and lysozyme (LYZ), excessive gaps in the sequence alignments were truncated based on a reference sequence. An apo form of E. coli malate dehydrogenase was used (PDB ID: 2PWZ) for MDH and the longest sequence in the MSA for LYZ (Uniprot ID: R6H6H5). Sequence weights, frequencies and positional correlations of amino acids were recomputed on the truncated alignment using pySCA package.

A main logic of the system 100 was expressed as a steering policy with Colmena. Colmena can define how to steer a scientific workflow as a series of agents which respond to changes in the workflow. For the evaluation of MDH and LYZ, a full protein design application can be composed of 5 agents that allow each of the processes to occur asynchronously yet that information from each agent can be propagated in a timely manner. The dynamic Colmena workflow can ensure that each task can be run at the desired time and with desired inputs.

The GenSLM models were evaluated on a diverse set of systems. The performance was explored on 1) Polaris supercomputer at the Argonne Leadership Computing Facility (ALCF), and 2) the Sunspot Test and Development system at ALCF—an early access system for the Aurora supercomputer. The performance was also evaluated on the SambaNova DataScale SN30 system.

The tested GenSLM was written with the PyTorch Lightning API, using transformer models from the Hugging Face repository. PyTorch Lightning allows the use of several distributed training strategies to scale model training on clusters and supercomputers. The training strategies can include DistributedDataParallel and DeepSpeed. The training runs used mixed precision using FP16 and FP32. FP16 and FP32 can refer to 16-bit and 32-bit floating point formats, DeepSpeed was focused on, as its employment of various ZeRO strategies for optimization reduces the overall memory utilization in model training, particularly for large parameter models. ZeRO strategies can be partition memory for training models—including the optimizer, gradient, and model states—to use aggregate memory across all GPUs. Using aggregate memory across all GPUs can enable training larger models on GPU-based systems and trades overall memory capacity for additional re-computation and communication. In particular, ZeRO-1 partitions optimizers across GPUs, ZeRO-2 partitions both the optimizers and gradients across all GPUs, and ZeRO-3 partitions the parameters, in addition to ZeRO-2 optimizations, across all GPUs. Additionally, ZeRO-3 can scale model sizes by leveraging CPU memory and any node-local storage to offload optimizer states, gradients, parameters, and optionally activations to CPU. PyTorch 1.12.0 and NVIDIA NCCL 2.10.3 were used as the backend for DeepSpeed. Aa bare-metal build using Conda on Polaris was using. On SN30, the implementation can be based on the Pytorch-based SambaFlow™ framework and uses bfloat16 precision.

Polaris has a peak of 44 PFLOPS. The Polaris system can be an HPE Apollo Gen10+ system with 560 nodes interconnected with HPE Slingshot 10 using a Dragonfly topology. Each node consists of an AMD “Milan” processor with 32 cores with 512 GB of system memory. Each node has four NVIDIA A100 GP Us—each with 40 GB memory. Each node has two Slingshot-10 endpoints at 12.5 GB/s for the interconnect network.

The Sunspot Test and Development System (TDS) consists of 2 racks, each with 64 nodes, for a total of 128 nodes Each node consists of 2× Intel Xeon CPU Max Series (codename Sapphire Rapids or SPR) and 6× Intel Data Center GPU Max Series (codename Ponte Vecchio or PVC). Each Xeon has 52 physical cores supporting 2 hardware threads per core Interconnect can be provided via 8×HPE Slingshot-11 NICs per node.

Pre-training performance was also evaluated for the PATRIC genomic sequences on the SambaNova (SN) DataScale® SN30 that has a total of 8 SambaNova Reconfigurable Dataflow Unit™ (RDUs) per node and 8 tiles per RDU. The SN30 uses a dataflow execution model with each RDU having 1280 programmable compute units, more than 600 MB on-chip memory, and 1 TB off-chip memory.

Selene can be based on the NVIDIA DGX SuperPOD platform and consists of 560 nodes interconnected with Mellanox HDR fabric. Each node consists of two AMD “Rome” processors, each with 64 cores and 2 TB system memory. Each node has eight NVIDIA A100 GPUs, each with 80 GB memory. Each node has eight Mellanox ConnectX-6 HDR endpoints at 20 GB/s each for the interconnect network. Each A100 NVIDIA GPU can be capable of achieving a peak of 19.5 TFLOPS in FP32, 156 TFLOPS in TF32, and 312 TFLOPS in FP16 and BF16.

To measure compute performance of GenSLM model training, the DeepSpeed flops profiler was used. The DeepSpeed flops profiler provides flops and latency of forward and backward passes and latency of weight updates, and thus a compute performance of the GenSLM models. For scaling studies, the end-to-end time including I/O as well as model training at scale was measured. Achieved throughput in samples per second as the number of GPUs scales on the system was measured. The NVIDIA Nsight tool to get an in-depth performance analysis was used.

FIG. 5 depicts the type of work being run on each GPU of Polaris over time, where GPUs are re-tasked between generating and scoring sequences or between different types of MD simulations. FIG. 5 depicts task being run on each node of Polaris over time. The nodes are ordered such that low-index nodes are those used for generation and scoring tasks and high-index nodes are those used for evaluating sequence performance. FIG. 5 also depicts the RMSF of each sequence over time. The sequence was run sequences from an increasingly well-trained model overtime, yielding proteins with increasingly better stabilities.

### EXAMPLES

The performance of scaling GenSLM training was evaluated on the Polaris, Sunspot, and SambaNova SN30 systems. Two target sequence lengths of 1,024 and 2,048 were used in scaling studies. In various runs, one rank was used per GPU with DeepSpeed ZeRO-2 optimizations on Polaris and Sunspot. On SN30, the GenSLM 13B and 25B models were trained using the SN reference implementation of GPT-2 model with a sequence length of 1024 that fits on 4 tiles or half RDU. Data parallelism was used to scale across multiple tiles and nodes. End-to-end execution times, throughput, and accuracy were measured. The batch size per GPU constant and scaled the global batch size appropriately was kept constant and scaled a global batch size appropriate as the number of GPUs was scaled. The learning rate parameter to account for scaling the number of ranks was modified. The performance obtained was the average of the throughput measured over multiple iterations.

The model size can increase from 13B to 25B, the total achievable throughput, in terms of samples/sec, decreases. The model size increasing can be expected as increasing the model size increases the computational, memory, and communication usage. In terms of efficiency, for smaller models, such as 25M, a drop in scaling efficiency was observed as the model scaled beyond 256 GPUs. Two key attributes can contribute to the drop in scaling efficiency including the fact that for smaller model sizes that run with ZeRO-3, the ratio of data movement to computational flops can be too high to completely overlap these. Better performance efficiency for larger models can be seen as they have higher utilization of computation and are able to better overlap communication with computation. Some inefficiencies here can also be due to a performance of collectives. In the case of the 25B model, a single batch was fitted on the GPU and a 50% improvement was observed in the throughput achieved on Selene over Polaris. The improvement can be attributed to an increased interconnect performance on Selene together with the larger memory capacity. A super-linear speedup was observed for the 25B case on both systems as the scale of the model increased to 1024 GPUs in comparison to the performance at 8 GPUs. The super-linear speedup can be attributed to an increased memory and data movement overheads at smaller GPU scales.

To gain detailed insights on the runs, a profiling study was performed on the 25M parameter model on both Polaris and Selene systems using the NVIDIA Nsight tool. To account for the difference in the number of GPUs in a single node on both systems, profiling runs were performed with the 32 GPUs on 8 nodes on Polaris and 4 on Selene separately. No significant delay was observed between the steps/iterations-data loading and I/O were not bottlenecks. Given that the Selene DGX node has 80 GB memory compared with 40 GB on the Polaris node, the batch size for the 25M parameter model can be doubled, thereby achieving higher throughput than Polaris.

In addition, a study was performed comparing the scaling behavior of the distributed training framework implementations for PyTorch DistributedDataParallel (DDP) and with DeepSpeed with ZeRO Stage 2 and 3 on Selene. With DDP, smaller model sizes can be used and may not employ any memory optimization, unlike DeepSpeed. DDP-based runs exhibit linear behavior, while the performance of DeepSpeed runs saturated beyond 256 GPUs for the ZeRO-2 optimizer and 512 GPUs for the ZeRO-3 optimizations. For the 25M case, at 512 GPUs, DDP achieves 99% scaling efficiency with a 10% improvement over ZeRO-3 and a 2× improvement over ZeRO-2. The scaling efficiency can be attributed to the fact that DDP implements AllReduce collective communication while DeepSpeed implements Reduce-Scatter and AllGather collective communication operations. The performance of the NCCL backend can be highly optimized for AllReduce in comparison to AllGather. The performance of the NCCL highlights an opportunity to explore further optimizations for the DeepSpeed implementation to scale on systems. There can be additional tuning knobs at the NCCL layer and in DeepSpeed.

For sequence length 10,240, a batch size of 1 and ZeRO-3 was used. The sequence length was increased from 2,048 to 10,240, and the memory usage, including for activation and residuals, increased by a similar factor. The computation usage can also grow by 5 times. One batch was able to be fit for the sequence length of 10,240 on the GPU with the current stages employed. At 512 GPUs, for the 25M case, a 50% improvement on Selene (64 nodes) over Polaris (128 nodes) can be observed. For the 250M case, an 11% improvement can be observed for Selene over Polaris. As the model size increased for the sequence length of 10,240, the model can be bottlenecked primarily by the memory subsystem performance and the overheads associated with staging residuals and parameters between the GPU and host.

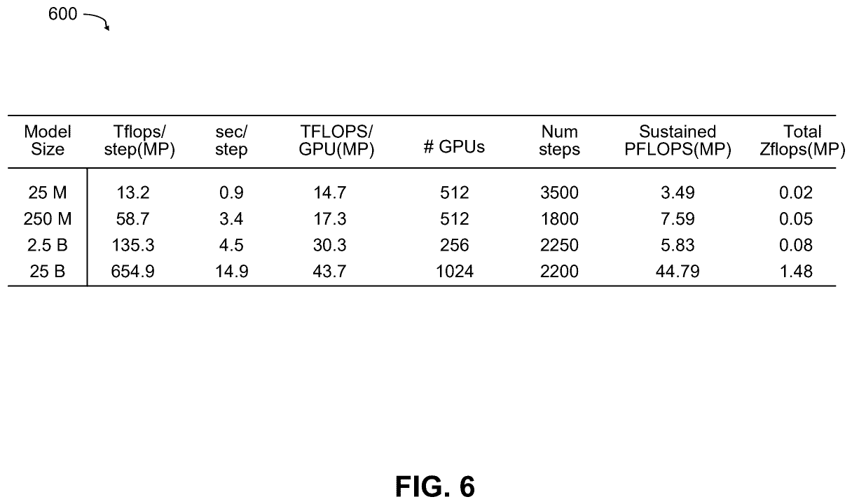

FIG. 6 is a table 600 of compute performance of the production runs in mixed precision (MP) for different model sizes with a sequence length of 2,048. The compute performance can include the I/O, computations for forward pass, backward pass, weight updates, and communication.

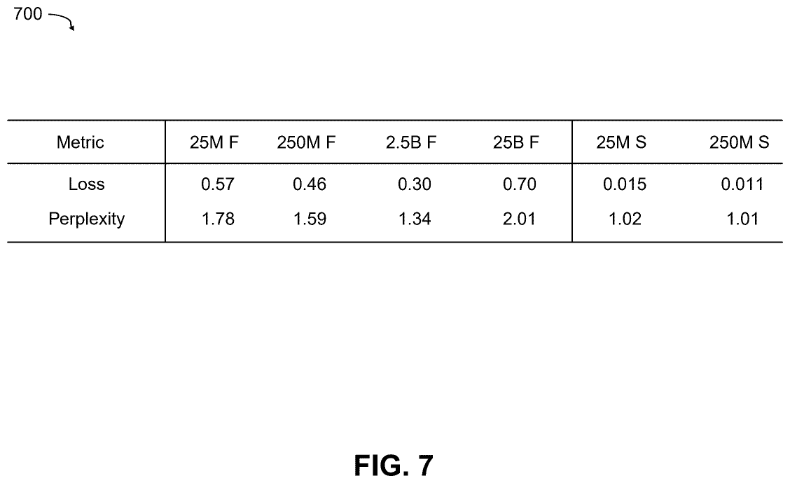

FIG. 6 illustrates the measured GPU performance obtained using the DeepSpeed profiler for smaller-scale runs. The efficiency of the runs was weak scaled to larger nodes and GPU counts for the sustained PFLOPS. The end-to-end application run, including data processing and checkpointing was accounted for. The number of GPUs for our production science runs on Polaris was chosen based on system availability, and the number of steps run was chosen to achieve an appropriate loss scale. As the model size was scaled, the overall computational flops per step increased given the increase in model complexity. For the 25B model, a sustained performance of 44.79 PFLOPS was achieved in mixed precision (MP). A peak performance of 212.55 PFLOPS(MP) was measured by accounting for the highest FLOPS consumed by a single layer in our network. For production science runs, an aggregate of 1.63 Zettaflops was used, and a 25B model used 1.48 Zettaflops to train on 1,024 GPUs for 2200 steps. For scaling runs on Selene with the 25B model, the runs can be scaled to 512 nodes with 4096 GPUs. A sustained performance of 121.26 PFLOPS(MP) and a peak performance of 850.21 PFLOPS(MP) were achieved. The final model performance can be seen in FIG. 7.

FIG. 7 is a table 700 of final loss and perplexity values achieved by the GenSLM Foundation (F) and SARS-CoV-2 (S) (10,240 tokens) models. Reported values for S models are trained on the first year of SARS-CoV-2 genomes. Perplexity can be computed by taking the exponential of the loss and can be interpreted as the number of guesses for the model to correctly fill a masked token.

The Sambanova Datascale SN30 Scalability was measured. The throughput and training time on the SN30 datascale engine for the GenSLM 13B and 25B with a sequence length of 1024 codon tokens for these scalability runs were measured. The SN30 can be scaled for a maximum of 8 SN30 nodes (which corresponds to 128 instances of data-parallel runs) with each instance having a micro batch size of 4. The large compute and on-chip memory of SN30 nodes can be leveraged to be run with the larger sequence length. A 13B GPT2 model was run for a sequence length of 32K and a throughput of 0.101 samples per second can be observed when scaled for 2 nodes and 0.319 samples per second for 8 nodes.

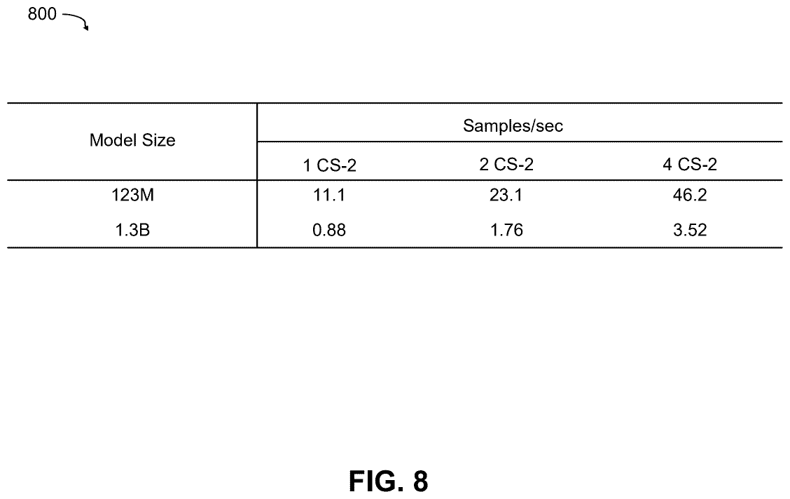

The Cerebras Wafer-Scale Cluster Scalability was measured. The throughput and training time of a Wafer-Scale Cluster for GenSLM-123M and GenSLM-1.3B with a sequence length of 10,240 codon tokens can be measured as depicted in FIG. 8. FIG. 8 is a table 800 of the Cerebras Wafer-Scale Cluster throughput training GenSLMs on a sequence length of 10,240 tokens. The batch size per CS-2 for each model was chosen based on empirical experiences with models of similar sizes and was kept constant when scaled up to multiple CS-2s. FIG. 8 shows average samples per second training GenSLM-123M and GenSLM-1.3B for 200 steps using one, two, and four CS-2s. Regardless of the model configurations, linear weak scaling in response to using up to four CS-2s was observed.

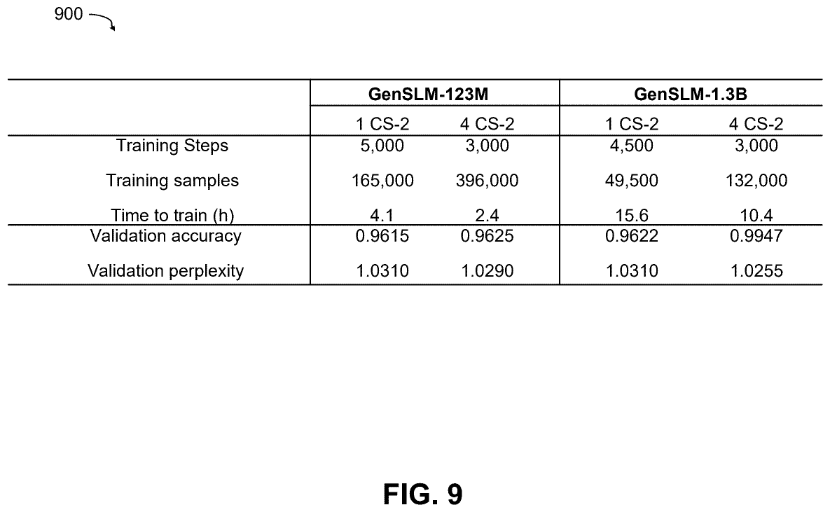

FIG. 9 is a table 900 of metrics of GenSLMs trained on a sequence length of 10,240 using Cerebras Wafer-Scale Cluster. GenSLM-123M and GenSLM-1.3B was trained from scratch using learned positional embeddings. FIG. 9 shows training time and a total number of training samples used to achieve validation accuracy >96% and perplexity <1.03 using one CS-2 and with four CS-2s. Validation measurements were taken from checkpoints every 500 steps. For GenSLMs of the same size, fewer training steps can be used to achieve comparable validation results in response to the global batch size being increased in a four-CS-2 Wafer-Scale Cluster with data parallelism. The reduced number of training steps plus linear weak scaling led to a reduction of at least a third of the training time in response to using four CS-2s versus one. All GenSLM training with full genomes on CS-2s converged within 12 hours. GenSLM-1.3B can use fewer training samples than the smaller GenSLM-123M to achieve comparable validation metrics, following the sample efficiency observation in neural language model scaling laws.

Performance of the workflow was measured. The key performance metric of the workflow can be how well (e.g., a degree, percentage) that the workflow keeps nodes allocated with work. The dynamic nature of our workflow means that high utilization cannot be relied on by preloading each node with much work. The workflow can include waiting as long as possible until deciding which protein can be worth assessing for stability. For example, the workflow can allow for idle time to determine the stability of the protein. Even with this “just in time” approach, there can be a median of 50 ms of downtime for our simulation tasks (thousands per hour) and only 30 seconds for the least frequent tasks (training). These idle times amount to 0.37% of the total runtime after the first simulations start at 15 minutes—a 99.6% system utilization during the run.

Beyond keeping nodes busy, many performance optimizations were made to ensure the work are as-fast as possible. One step can be to keep the machine learning models used for folding in memory be-tween tasks, visible in the first folding tasks which can be 15% longer to complete than all others. ProxyStore were employed separately from workflow engine control messages. The application can use Redis as a data fabric for Proxystore, which results in data transfer rates in excess of 1 GB/s and keeps workflow engine latencies to below is even for the largest task outputs.

The steering process of Colmena may not be overwhelmed by the scale of the runs. Scaling a dynamic workflow to full-machine scales can involve being able to make task decisions at high rates. The Colmena steering agents achieves a Is-sustained task submission rate of 258 tasks/second during the early stages of the run in response to the initial simulation tasks starting. The rate of task completion can be on the order of thousands per hour, which can be below the observed capacity of Colmena. The simulation tasks, which are the most frequent processed tasks in the application, can use 4 ms to process each. As seen in the 99% utilization, these quick response times are quite sufficient to keep the system busy.

In overview, the workflow can fully leverage Polaris. The workflow can keep workers busy, mitigate the cost of loading large machine learning models, and utilize the fast interconnects to manage large data produced by the workflow. Given the high utilization and available decision-making throughput from Colmena, the workflow and application can be able to deploy our protein design engine on even larger supercomputers or, even, across multiple computing resources.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein may be embodied in other specific forms without departing from the characteristics thereof. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

In various implementations, the steps and operations described herein may be performed on one processor or in a combination of two or more processors. For example, in some implementations, the various operations could be performed in a central server or set of central servers configured to receive data from one or more devices (e.g., edge computing devices/controllers) and perform the operations. In some implementations, the operations may be performed by one or more local controllers or computing devices (e.g., edge devices), such as controllers dedicated to and/or located within a particular building or portion of a building. In some implementations, the operations may be performed by a combination of one or more central or offsite computing devices/servers and one or more local controllers/computing devices. All such implementations are contemplated within the scope of the present disclosure. Further, unless otherwise indicated, when the present disclosure refers to one or more computer-readable storage media and/or one or more controllers, such computer-readable storage media and/or one or more controllers may be implemented as one or more central servers, one or more local controllers or computing devices (e.g., edge devices), any combination thereof, or any other combination of storage media and/or controllers regardless of the location of such devices.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

What is claimed is:

1. A method, comprising:
generating, by one or more processors using a machine learning model, based on an initial protein sequence data structure, a plurality of protein sequences, the machine learning model configured based on reinforcement learning from a plurality of reward metrics including at least one reward metric associated with experimental data regarding example sequence data;
scoring, by the one or more processors, using a plurality of scoring functions, the plurality of protein sequences, to select a subset of protein sequences of the plurality of protein sequences; and
outputting one or more selected protein sequences of the subset of selected protein sequences.

2. The method of claim 1, wherein:
the machine learning model comprises a language model; and
determining, by the machine learning model, each protein sequence of the plurality of protein sequences comprises generating, by the language model, one or more protein sequence elements based on the initial protein sequence data structure.

3. The method of claim 1, wherein the machine learning model is configured based on reinforcement learning by a plurality of agents, each agent of the plurality of agents associated with a different reward metric of the plurality of reward metrics than each other agent of the plurality of agents.

4. The method of claim 1, wherein the machine learning model comprises a pre-trained language model fine-tuned based on the plurality of reward metrics.

5. The method of claim 1, wherein the plurality of scoring functions comprise at least a similarity function based on a database of example sequence data, a folding function, and a stability function, and the method comprises scoring using the stability function responsive to an output of at least one of the similarity function or the folding function satisfying a corresponding threshold.

6. The method of claim 1, wherein generating the plurality of protein sequences comprises generating a plurality of protein sequence elements for each protein sequence of the plurality of protein sequences, wherein each protein sequence element of the plurality of protein sequence elements represents at least one of a codon or a protein residue.

7. The method of claim 1, wherein the plurality of scoring functions comprise at least one function based on at least one of a guanine-cytosine (GC) content or a molecular weight of each protein sequence of the plurality of protein sequences.

8. The method of claim 1, wherein the plurality of reward metrics comprise at least one evolutionary conservation metric.

9. The method of claim 1, wherein the plurality of reward metrics comprise at least one molecular simulation metric.

10. The method of claim 1, further comprising asynchronously performing, by the one or more processors in parallel using a plurality of parallel computing resources, at least one of the generating of the plurality of protein sequences or the scoring of the plurality of protein sequences.

11. The method of claim 1, wherein the plurality of scoring functions comprise an activity function to determine an activity of at least one protein sequence of the plurality of protein sequences.

12. A system, comprising:
one or more processors to:
generate, using a machine learning model, based on an initial protein sequence data structure, a plurality of protein sequences, the machine learning model configured based on reinforcement learning from a plurality of reward metrics including at least one reward metric associated with experimental data regarding example sequence data;
score, using a plurality of scoring functions, the plurality of protein sequences, to select a subset of protein sequences of the plurality of protein sequences; and
output one or more selected protein sequences of the subset of selected protein sequences.

13. The system of claim 12, wherein:
the machine learning model comprises a language model; and
the one or more processors are to determine each protein sequence of the plurality of protein sequences by generating, using the language model, one or more protein sequence elements based on the initial protein sequence data structure.

14. The system of claim 12, wherein the machine learning model is configured based on reinforcement learning by a plurality of agents, each agent of the plurality of agents associated with a different reward metric of the plurality of reward metrics than each other agent of the plurality of agents.

15. The system of claim 12, wherein:
the machine learning model comprises a pre-trained language model fine-tuned based on the plurality of reward metrics; and
the plurality of reward metrics comprise at least one evolutionary conservation metric and at least one molecular simulation metric.

16. The system of claim 12, wherein the plurality of scoring functions comprise at least a similarity function based on a database of example sequence data, a folding function, and a stability function, and the plurality of scoring functions further comprises scoring using the stability function responsive to an output of at least one of the similarity function or the folding function satisfying a corresponding threshold.

17. The system of claim 12, wherein the one or more processors comprise a plurality of parallel processing units to asynchronously perform at least one of the generation of the plurality of protein sequences or the scoring of the plurality of protein sequences.

18. A method, comprising:
generating, by each of a plurality of reinforcement learning agents, for each protein sequence of a plurality of examples of protein sequences, a reward score for an objective function, the reward score generated based on a different metric for each agent of the plurality of reinforcement learning agents;
evaluating the objective function using each reward score to generate an output of the objective function; and
updating a language model based on the output.

19. The method of claim 18, wherein a metric of a first agent of the plurality of reinforcement learning agents corresponds to a structure of the protein sequence, and the metric of a second agent of the plurality of reinforcement learning agents corresponds to kinetics of the protein sequence.

20. The method of claim 18, wherein updating the language model comprises evaluating a Kullback-Leibler divergence with respect to a previous state of the language model.
